SEQUENCE LISTING (7 sequences)

SEQ ID NO: 1 - DNA, 2244 bp, Artificial Sequence
  torA-SbAmyA consisting of TorA sequence expressing torA signal peptide and SbAmyA sequence expressing alpha-amylase
catatgaacaataacgatctctttcaggcatcacgtcggcgttttctggcacaactcggcggcttaaccgtcgccgggatgctggggccgtcattgttaacgccgcgacgtgcgactgcggcgcaagcggcggatgaacaggtgtccatgaaggatggcaccatcctgcacgcatggtgctggtccttcaacaccatcaaggataacatgcaggcaatcaaggatgcaggctacacctccgtgcagacctccccaatcaacaccgtggtggcaggcgaaggcggcaacaagtccctgaagaactggtactaccagtaccagccaaccatctacaagatcggcaactaccagctgggcaccgaagaagaatttaaggaaatgaaccgcgtggcagatcagtacggcatcaagatcatcgtggatgcagtgctgaaccacaccacctccgattacaaccagatttcccaggaaatcaagaacatcccaaactggacccacggcaacaccctgatctccgattggcacaaccgctacgatgtgacccagaacgcactgctgaccctgtacgattggaacacccagaacgaatacgtgcagcagtacctgctgtcctacctgaagcaggcagtggcagatggcgcagatggcttccgctacgatgcagcaaagcacatcgaactgccaggcgaatacggctccaacttctggaacgtgatcctgaacaacggctccgaatttcagtacggcgaaatcctccaggatgatgtgtccaacgatgcaggctacggcaagctgatgtccatcaccgcatccaactacggccagaagatccgctccgcactgaaggatcgccacatctccgcaggcaacctgatgaactaccaggtgtccggcgtggatgcagcaaacctggtgacctgggtggaatcccacgataactacgcaaacgatgatcaggaatccacctggatgaacgattccgatatcggcctgggctgggcaatgatcaccgcacgcgcaaagggcaccccactgttcttctcccgcccagtgggcggcggcaacggcacccgcttcccaggccagtcccagatcggcgatgcaggctccaacctgtacaaggatgcaaccgtgaccgcagtgaacaagttccacaacgcaatggtgggcgaatccgaatacctgcgcaacccaggcggcgatgaacaggtggcaatgatcgaacgcggcaccaagggcgcagtgatcgtgaacctggtggatggcgataagcagatcaactccgaaaccaacctggcagatggcacctacaccgataaggtgtccggccgccagttcaacgtgtccaacggccgcatcaccggctccgtgccatcccgctccgcagtggtgctgtacgatgatcaggcatcccaggcagcacaggtgtccgtggatggctacaaggaaggcgataactccatctccaaggcaaccgaagtgaccctgaaggcaaagaacgcagattccgcaacctacaagctgggcaacggccaggaagtggcatacaaggatggcgataaggtgaccgtgggcgaaggcctggaagcaggccagtccaccaccctgaccctgaccgcaaccggcgcagatggccagtccaccaccaagacctacaccttcaccatgaaggacccatccgcagaaaccaacatctacttccagaacccagataactggtccgaagtgtacgcatacatgtactccgcaaaggataacaagctgctgggcgcatggccaggcaccaagatgaccaaggaagcatccggccgctactccatcaccgtgccagcatcctacgcagaagaaggcgtgaaggtgatcttcaccaacaaccagggctcccagtacccacagaacgaaggcttcgatttcaaggcagaaggcctgtactccaaggcaggcctgatgccagatgtgccagcaggcaagacccgcgtgaccttcgataacccaggcggctgggattccgcaaacgcatacctgtactacggcaacccagtgcagtacccactgggcgtgtggccaggcacccagatgaccaaggatgatgcaggcaacttctacctggatctgccagaagaatacgcagatgtgaacgcaaagatcatcttcaaccagccaggcacctccaaccagtacccatactccgaaggcttcaacctggtgaagtccggcaactacaacaaggatggcctgaagtaa

SEQ ID NO: 2 - DNA, 1584 bp, Artificial Sequence
  torA-BlAmyS consisting of TorA sequence expressing torA signal peptide and BlAmyS sequence expressing alpha-amylase
catatgaacaataacgatctctttcaggcatcacgtcggcgttttctggcacaactcggcggcttaaccgtcgccgggatgctggggccgtcattgttaacgccgcgacgtgcgactgcggcgcaagcggcggcaaacctgaacggcaccctgatgcagtacttcgaatggtacatgccaaacgatggccagcactggaagcgcctccagaacgattccgcatacctggcagaacacggcatcaccgcagtgtggattccaccagcatacaagggcacctcccaggcagatgtgggctacggcgcatacgatctgtacgatctgggcgagttccaccagaagggcaccgtgcgcaccaagtacggcaccaagggcgaactccagtccgcaatcaagtccctgcactcccgcgatatcaacgtgtacggcgatgtggtgatcaaccacaagggcggcgcagatgcaaccgaagatgtgaccgcagtggaagtggacccagcagatcgcaaccgcgtgatctccggcgaacaccgcatcaaggcatggacccacttccacttcccaggccgcggctccacctactccgatttcaagtggcactggtaccacttcgatggcaccgattgggatgaatcccgcaagctgaaccgcatctacaagttccagggcaaggcatgggattgggaagtgtccaacgaaaacggcaactacgattacctgatgtacgcagatatcgattacgatcacccagatgtggcagcagaaatcaagcgctggggcacctggtacgcaaacgaactccagctggatggcttccgcctggatgcagtgaagcacatcaagttctccttcctgcgcgattgggtgaaccacgtgcgcgaaaagaccggcaaggaaatgttcaccgtggcagaatactggtcctacgatctgggcgcactggaaaactacctgaacaagaccaacttcaaccactccgtgttcgatgtgccactgcactaccagttccacgcagcatccacccagggcggcggctacgatatgcgcaagctgctgaactccaccgtggtgtccaagcacccactgaaggcagtgaccttcgtggataaccacgatacccagccaggccagtccctggaatccaccgtgcagacctggttcaagccactggcatacgcattcatcctgacccgcgaatccggctacccacaggtgttctacggcgatatgtacggcaccaagggcgattcccagcgcgaaatcccagcactgaagcacaagatcgaaccaatcctgaaggcacgcaagcagtacgcatacggcgcacagcacgattacttcgatcaccacgatatcgtgggctggacccgcgaaggcgattcctccgtggcaaactccggcctggcagcactgatcaccgatggcccaggcggcgcaaagcgcatgtacgtgggccgccagaacgcaggcgaaacctggcacgatatcaccggcaaccgctccgaaccagtggtgatcaactccgaaggctggggcgagttccacgtgaacggcggctccgtgtccatctacgtgcagcgctaa

SEQ ID NO: 3 - DNA, 132 bp, Artificial Sequence
  torA sequence which expresses torA signal peptide
catatgaacaataacgatctctttcaggcatcacgtcggcgttttctggcacaactcggcggcttaaccgtcgccgggatgctggggccgtcattgttaacgccgcgacgtgcgactgcggcgcaagcggcg

SEQ ID NO: 4 - DNA, 6808 bp, Artificial Sequence
  vector pBbEB1c-rfp
gacgtcgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcagaattcaaaagatcttttaagaaggagatatacatatggcgagtagcgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaaggatccaaactcgagtaaggatctccaggcatcaaataaaacgaaaggctcagtcgaaagactgggcctttcgttttatctgttgtttgtcggtgaacgctctctactagagtcacactggctcaccttcgggtgggcctttctgcgtttatacctagggcgttcggctgcggcgagcggtatcagcagttattggtgcccttcgaaatgaccgaccaagcgacgcccaacctgccatcacgagatttcgattccaccgccgccttctatgaaaggttgggcttcggaatcgttttccgggacgccaacaacaagacccatcatagtttgcccccgcgacattgaccataaattcatcgcacaaaatatcgaacggggtttatgccgcttttagtgggtgcgaagaatagtctgctcattacccgcgaacaccgccgcattcagatcacgcttagtagcgtccccatgagtaggcagaaccgcgtccaagtccacatcatccataacgatcatgcacggggtggaatccacacccagacttgccagcacctcattagcgacacgttgcgcagcggccacgtccttagccttatccacgcaatcgagaacgtactgcctaaccgcgaaatcagactgaatcagtttccaatcatcgggcttcaccaaagcaacagcaacgcgggttgattcgacccgttccggtgcttccagaccggcgagcttgtacagttcttcttccatttcacgacgtacatcagcgtctatgtaatcaatgcccaaagcacgcttagccccacgtgaccaggacgaacgcaggtttttagaaccaacctcatactcacgccaccgagccaccaaaacagcgtccatatcctcgccggcgtcgctttgatcggccaacatatccaacatctgaaacggcgtgtacgaccccttagacgcggttttagtagcggagccagtcagttcctgagacatgcccttagcgaggtaggttgccattttcgcagcgtctccaccccaggtagacacctgatcaagtttgaccccgtgctcacgcagtggcgcgtccataccggccttaaccacaccagcagaccagcgggaaaacatggaatcctcaaacgccttgagttcatcgtcagacagtggacgatccaagaacaacagcatgttgcggtgcaagtgccaaccgttcgcccaagagtctgtgacctcatagtcactataggtgtgctccaccccgtaccgtgcacgttctttcttccactgagatgttttcaccatcgaagagtacgcagtcttaatacccgcttcaacctgcgcaaatgactgtgagcggttgtgtcgaacagtgcccacaaacatcatgagcgcgccacccgccgccaagtgattcttagtagcaatagccagctcaatgcggcgttcgcccatgacttccaattcagccagaggtgacccccagcgagagtgagagttttgcagaccctcaaactgcgaagcaccgttagacgaccaggacaccgcaacagcttcgtccctgcgccacctatggcaccccgccagagccttactattggtgatcttgtacatgacgttttgcctacgccacgccctagcgcgagtgaccttagaaccctcattgacctgcggttccttagaggtgttcacttctatttcagtgttactcagtgttacctagacccgatgttgtgcggggttgcgcagtgcgagtttgtgcgggtgttgtgcccgttgtcttagctagtgctatggttgtcaattgaaaccccttcgggttatgtggcccccgtgcatatgagttggtagctcgcacgggggtttgtcttgtctagggactattaatttttagtggtgtttggtggccgcctagcttggctatgcgtgccagcttacccgtactcaatgttaaagatttgcatcgacatgggagggttacgtgtccgatacctagggggggtatccgcgactaggtgccccggtgctcactgtctgtaccggcggggcaagccccacaccccgcatggacagggtggctccgccccctgcacccccagcaatctgcatgtacatgttttacacattagcacgacatgactgcatgtgcatgcactgcatgcagactaggtaaatatgagtatgtacgactagtaacaggagcactgcacataatgaatgagttgcaggacaatgtttgctacgcatgcgcatgacatatcgcaggaaagctactagagtcttaaagcatggcaaccaaggcacagctagaacagcaactacaagaagctcaacaggcactacaggcgcagcaagcgcaggcacaagccaccatcgaagcactagaagcgcaggcaaaggctaagcccgtcgtggtcaccgcacgcgttcctttggcactacgtgaggacatgaagcgcgcaggcatgcagaacggtgaaaacctccaagagttcatgatcgccgcgtttaccgagcggctagaaaagctcaccaccaccgacaacgaggaaaacaatgtctaacccactagttctctttgcccaccgtgacccggtaaatgacgtgacgttcgagtgcattgagcacgccacctacgacacactttcacacgctaaagaccagatcaccgcccaaatgcaagccctagacgaagaagccgccctactgccctaatgggtgtttcatgggtgtttccctagtgtttcatggtgttttcacctaagctagggaattgcgcgagaagtctcgcaaaaatcagcaacccccggaaccacacagttcacgggggttcttctatgccagaaatcagaaaggggaaccagtgaacgaccccgaatggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatc

SEQ ID NO: 5 - DNA, 6 bp, Artificial Sequence
  EcoRI restrction enzyme
gaattc

SEQ ID NO: 6 - DNA, 6 bp, Artificial Sequence
  BamHI restrction enzyme
ggatcc

SEQ ID NO: 7 - DNA, 678 bp, Artificial Sequence
  rfp sequence of vector pBbEB1c-rfp
atggcgagtagcgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaa

(19) United States
(12) Patent Application Publication
(10) Pub. No.: US 2016/0002677 A1
(43) Pub. Date: Jan. 7, 2016

(54) STRAIN FOR PRODUCING SUCCINATE FROM CARBON DIOXIDE AND METHOD FOR SUCCINATE PRODUCTION USING THE STRAIN

(71) Applicant: KOREA INSTITUTE OF SCIENCE AND TECHNOLOGY, Seoul (KR)

(72) Inventors: Han Min WOO, Seoul (KR); Jung Seok LEE, Goyang-si (KR); Youngsoon UM, Seoul (KR); Gyeong Taek GONG, Seoul (KR); Byoung Koun MIN, Seoul (KR); Jeong-Myeong HA, Seoul (KR); Yunje KIM, Seoul (KR); Sang Jun SIM, Seoul (KR)

(73) Assignee: KOREA INSTITUTE OF SCIENCE AND TECHNOLOGY, Seoul (KR)

(21) Appl. No.: 14/494,930

(22) Filed: Sep. 24, 2014

(30) Foreign Application Priority Data
Jul. 2, 2014 (KR) 10-2014-0082576

(51) Int. Cl. C12P 7/46; C12N 15/77

(52) U.S. Cl. CPC C12P 7/46; C12N 15/77

(57) ABSTRACT

The present disclosure relates to a strain capable of producing succinate using starch accumulated in microalgae which grow using carbon dioxide as a direct carbon source without converting it to glucose and a method for producing succinate using the same. The present disclosure provides a strain producing succinate from carbon dioxide, selected from a group consisting of Corynebacterium glutamicum BL-1-pBlAmyS (KCTC 12585BP) and Corynebacterium glutamicum BL-1-pSbAmyA (KCTC 12587BP). The present disclosure also provides a method for producing succinate from carbon dioxide, including fermenting starch by inoculating the strain producing succinate from carbon dioxide in a starch-containing medium.

### CROSS-REFERENCE TO RELATED APPLICATION

This application claims priority to Korean Patent Application No. 10-2014-0082576, filed on Jul. 2, 2014, and all the benefits accruing therefrom under 35 U.S.C. §119, the contents of which in its entirety are herein incorporated by reference.

### BACKGROUND

1. Field

The present disclosure relates to a method for genetically engineering a succinate-producing strain to use a microalgal biomass as a carbon source and a method for producing succinate from carbon dioxide using the strain.

[Description about National Research and Development Support]

This work was supported by the National Research Foundation of Korea grant-funded by the Korean Government (Ministry of Science, ICT & Future Planning, Republic of Korea) (2014, University-Institute cooperation program) (Project No. 1711008615). Also, this study was supported by KCRC CCS2020 Project of Ministry of Science, ICT and Future Planning, Republic of Korea (Development of orginal technology using recombinated cyanobacteria for continuously direct-producing of biodiesel, Project No. 2014M1A8A1049277) under the superintendence of Korea Institute of Science and Technology.

2. Description of the Related Art

As global warming becomes a severe problem, use of microalgae to reduce carbon dioxide (CO₂) in the atmosphere is gaining attentions. Microalgae are currently variously utilized in the production of biodiesel and development of cosmetic materials, medicine and functional materials. Microalgae can grow into renewable and sustainable biomass in the presence of water and light, thus accumulating various carbohydrates including starch in cells. For this reason, researches are actively under way on production of various chemical products using the microalgal biomass that can replace lignocellulosic biomass which is problematic due to high cost and low abundance.

Corynebacterium glutamicum is a bacterial species producing various amino acids and nucleic acids and is widely used industrially at present. Although the wild-type Corynebacterium glutamicum can use glucose and sucrose as carbon sources, it cannot utilize xylose, cellobiose and starch. Therefore, a process of saccharification is necessary for fermentation of the lignocellulosic biomass by Corynebacterium glutamicum. But, there are problems that the lignocellulosic biomass in a manner of renewable resource is slower than microalgal biomass and fermentation inhibitors may be produced during the saccharification process.

Korean Patent Registration Publication No. 10-1339960, which relates to a microorganism capable of producing organic acids using algae in the family Hydrodictyaceae as biomass, describes use of a microorganism other than Corynebacterium glutamicum. However, this patent also requires a saccharification process using an enzyme. US Patent Publication No. 2012-0315678 discloses to a method for fermenting microalgal biomass using a microorganism. However, the method also requires an additional pretreatment process of converting starch accumulated in microalgae to glucose.

### REFERENCES OF THE RELATED ART

### Patent Documents

Korean Patent Registration Publication No. 10-1339960 (Dec. 4, 2013).
US Patent Publication No. 2012-0315678 (Dec. 13, 2012).

### Non-Patent Documents

“Efficient aerobic succinate production from glucose in minimal medium with Corynebacterium glutamicum mbt_{—}310 116 . . . 1”, Litsanov et al., Microbial Biotechnology (2012) 5(1), 116-128.

### SUMMARY

The present disclosure is directed to providing a strain which is genetically engineered to produce succinate from starch accumulated in microalgae that grow using carbon dioxide as a direct carbon source without using a saccharification enzyme, a method for preparing the same and a method for producing succinate from carbon dioxide using the same.

In an aspect, the present disclosure provides a strain producing succinate from carbon dioxide, selected from a group consisting of Corynebacterium glutamicum BL-1-pBlAmyS (KCTC 12585BP) and Corynebacterium glutamicum BL-1-pSbAmyA (KCTC 12587BP).

In another aspect, the present disclosure provides a method for preparing the strain producing succinate from carbon dioxide, including: preparing torA-SbAmyA (SEQ ID NO 1) by attaching TorA signal peptide to the coding sequence of AmyA or preparing torA-BlAmyS (SEQ ID NO 2) by attaching TorA signal peptide to the coding sequence of AmyS; inserting the torA-SbAmyA (SEQ ID NO 1) or the torA-BlAmyS (SEQ ID NO 2) to a vector expressible in Corynebacterium glutamicum BL-1 as a mother strain and genetically recombining the same with the mother strain Corynebacterium glutamicum BL-1; and overexpressing the inserted torA-SbAmyA (SEQ ID NO 1) or torA-BlAmyS (SEQ ID NO 2) in the mother strain.

In another aspect, the present disclosure provides a method for producing succinate from carbon dioxide, including fermenting starch by inoculating the strain producing succinate from carbon dioxide in a starch-containing medium.

Since the strain producing succinate from carbon dioxide according to the present disclosure can produce succinate from carbon dioxide by fermenting microalgal biomass which biologically converts carbon dioxide and accumulates starch with fast carbon cycle, it can contribute to solve the global warming problem. Since it grows using the starch accumulated in the microalgal biomass as a direct carbon source, a pretreatment process of starch saccharification is unnecessary and production of fermentation inhibitors during the pretreatment process can be prevented. The present disclosure is economical because the microalgal biomass can be produced in large scale. The present disclosure can resolve the problem of the existing process of producing organic acids from crops through saccharification which is affected by the increase in crop prices and exhibits slow carbon cycle.

The succinate produced by the strain according to the present disclosure can be used in the production of highly value-added biochemical products such as polybutylene succinate (PBS) which is used as a food packaging material.

### BRIEF DESCRIPTION OF THE DRAWINGS

FIG. 1 schematically describes a process of producing succinate from carbon dioxide according to an exemplary embodiment of the present disclosure.

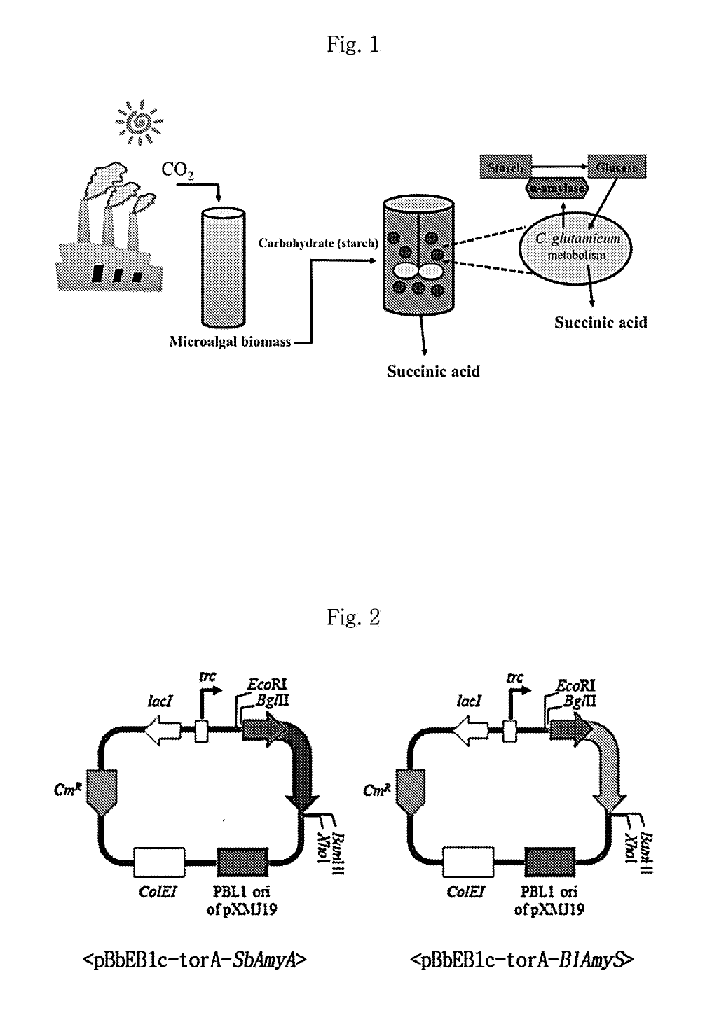

FIG. 2 shows torA-SbAmyA (SEQ ID NO 1) (left) and torA-BlAmyS (SEQ ID NO 2) (right) inserted into the pBbEB1c-rfp vector, which is an expression vector of Corynebacterium glutamicum, according to an exemplary embodiment of the present disclosure.

FIG. 3 shows a result of analyzing growth of wild-type Corynebacterium glutamicum and starch concentration in a minimal medium containing 0.5% starch (: OD, ▪: starch concentration).

FIGS. 4a-4c show a result of analyzing growth of Corynebacterium glutamicum ATCC13032 wherein only the expression vector pBbEB1c has been inserted (FIG. 4a) and Corynebacterium glutamicum ATCC13032 wherein the target gene AmyA (FIG. 4b) or the target gene AmyS (FIG. 4c) has been inserted and starch concentration in a medium containing 0.5% starch (: OD, ▪: starch concentration).

FIG. 5 shows a result of analyzing growth of succinate-producing strain Corynebacterium glutamicum BL-1-pBbEB1c wherein only the expression vector pBbEB1c has been inserted, Corynebacterium glutamicum BL-1-pSbAmyA wherein the target gene AmyA has been inserted and succinate-producing strain Corynebacterium glutamicum BL-1-pBlAmyS wherein the target gene AmyS has been inserted in a medium containing 0.5% starch and 0.5% glucose.

FIG. 6 shows a result of analyzing production of succinate by succinate-producing strain Corynebacterium glutamicum BL-1-pBbEB1c wherein only the expression vector pBbEB1c has been inserted, Corynebacterium glutamicum BL-1-pSbAmyA wherein the target gene AmyA has been inserted and succinate-producing strain Corynebacterium glutamicum BL-1-pBlAmyS wherein the target gene AmyS has been inserted in a medium containing 0.5% starch and 0.5% glucose.

FIG. 7 shows a result of analyzing growth of succinate-producing strain Corynebacterium glutamicum BL-1-pBbEB1c wherein only the expression vector pBbEB1c has been inserted, Corynebacterium glutamicum BL-1-pSbAmyA wherein the target gene AmyA has been inserted and succinate-producing strain Corynebacterium glutamicum BL-1-pBlAmyS wherein the target gene AmyS has been inserted in a minimal medium containing 0.2% of total sugar isolated from microalgal biomass.

FIG. 8 shows a result of analyzing production of succinate by succinate-producing strain Corynebacterium glutamicum BL-1 (BL-1-pBbEB1c) wherein only the expression vector pBbEB1c has been inserted, Corynebacterium glutamicum BL-1-pSbAmyA wherein the target gene AmyA has been inserted and succinate-producing strain Corynebacterium glutamicum BL-1-pBlAmyS wherein the target gene AmyS has been inserted in a medium containing 0.2% of total sugar isolated from microalgal biomass.

### DETAILED DESCRIPTION

Hereinafter, the present disclosure is described in further detail.

In an aspect, the present disclosure provides a strain producing succinate from carbon dioxide, selected from a group consisting of Corynebacterium glutamicum BL-1-pBlAmyS (KCTC 12585BP) and Corynebacterium glutamicum BL-1-pSbAmyA (KCTC 12587BP).

The strain producing succinate from carbon dioxide according to an exemplary embodiment of the present disclosure is a strain which grows using starch accumulated in microalgal biomass that grows using carbon dioxide as a direct carbon source and may be a strain prepared by the method described below.

Specifically, in an exemplary embodiment of the present disclosure, the strain producing succinate from carbon dioxide may be prepared by a method including: preparing torA-SbAmyA (SEQ ID NO 1) by attaching TorA signal peptide to the coding sequence of AmyA or preparing torA-BlAmyS (SEQ ID NO 2) by attaching TorA signal peptide to the coding sequence of AmyS; inserting the torA-SbAmyA (SEQ ID NO 1) or the torA-BlAmyS (SEQ ID NO 2) to a vector expressible in Corynebacterium glutamicum BL-1 as a mother strain and genetically recombining the same with the mother strain Corynebacterium glutamicum BL-1; and overexpressing the inserted torA-SbAmyA (SEQ ID NO 1) or torA-BlAmyS (SEQ ID NO 2) in the mother strain.

The Corynebacterium glutamicum BL-1 strain is a strain engineered from the Corynebacterium glutamicum ATCC13032 (Accession No. NC_{—}006958, Version NC_{—}006958.1 GI: 62388892) strain to be capable of producing succinate.

Information about the Corynebacterium glutamicum BL-1 strain can be found in “Efficient aerobic succinate production from glucose in minimal medium with Corynebacterium glutamicum mbt_{—}310 116 . . . 1”, Litsanov et al., Microbial Biotechnology (2012) 5(1), 116-128, which is herein incorporated by reference in its entirety. According to the literature, although the wild-type Corynebacterium glutamicum ATCC13032 strain may be engineered to produce succinate by removing the sdhCAB gene which encodes succinate dehydrogenase, in this case, acetate is also produced in large quantity as a byproduct. Also, it is described that the Corynebacterium glutamicum ATCC13032 can be engineered to produce succinate with high efficiency by removing the genes involved in acetate-producing pathways. However, the Corynebacterium glutamicum BL-1 strain needs conversion of starch to glucose, etc. to grow in biomass containing starch such as microalgae. In contrast, the present disclosure has resolved the problem of the existing Corynebacterium glutamicum BL-1 strain by providing a strain capable of producing succinate using starch-containing microalgal biomass as a carbon source by transforming the Corynebacterium glutamicum BL-1 strain with torA-SbAmyA (SEQ ID NO 1) or torA-BlAmyS (SEQ ID NO 2).

AmyA and AmyS are genes expressing α-amylase. The strain according to an exemplary embodiment of the present disclosure which includes the gene expressing α-amylase can grow using starch as a direct carbon source. The AmyA and AmyS can hydrolyze and convert soluble starch to glucose by expressing α-amylase (see Scheme 1). Since the TorA signal peptide, being attached to the coding sequence of AmyA and AmyS, secretes the α-amylase expressed by the AmyA and AmyS out of the cells the strain, succinate can be produced using starch-containing microalgal biomass as a carbon source. In an exemplary embodiment, the AmyA may be one derived from Streptococcus bovis and the AmyS may be one derived from Bacillus licheniformis.

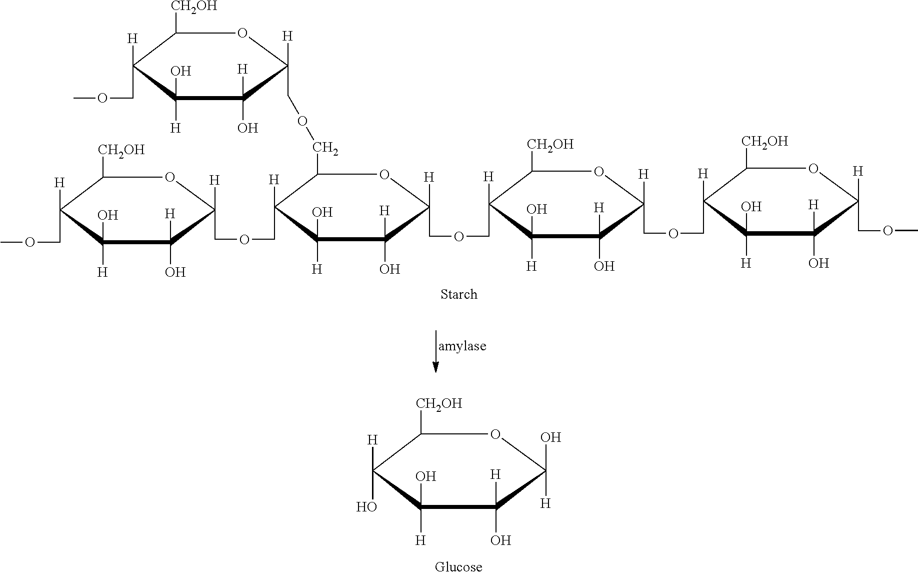

In an exemplary embodiment of the present disclosure, the strain producing succinate from carbon dioxide is a strain which grows using starch as a direct carbon source and produces succinate. In an exemplary embodiment, the starch is a starch accumulated in microalgal biomass. The microalgal biomass, in which various carbohydrates are accumulated in cells in addition to cellulose and lipid components, grows quickly. Specifically, the microalgae are known to be composed of about 60% of carbohydrate, about 30% of which being starch. The microalgae are not particularly limited as long as they accumulate starch. Specific examples may include Chlorella vulgaris, Chlorella sorokiniana, Chlorella sorokiniana, Chlamydomonas reinhardtii UTEX 90, etc. Since the strain producing succinate from carbon dioxide according to an exemplary embodiment of the present disclosure can use starch as a direct carbon source, the growth rate of strain can be about 3 times or more higher in a starch-containing medium as compared to the mother strain Corynebacterium glutamicum BL-1 not including AmyA or AmyS.

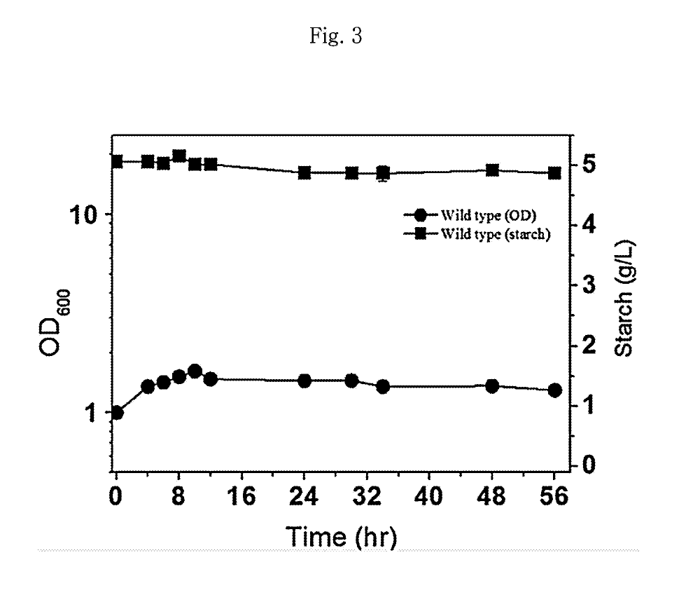

In an exemplary embodiment of the present disclosure, the vector expressible in Corynebacterium glutamicum BL-1 may be, for example, pBbEB1c-rfp (SEQ ID NO 4; Accession No. KJ021042, Version KJ021042.1 GI: 605098424). However, any one that can be used for expression of torA-SbAmyA (SEQ ID NO 1) or torA-BlAmyS (SEQ ID NO 2) in Corynebacterium glutamicum BL-1 may be used without limitation. FIG. 2 shows AmyA and TorA signal peptide (torA-SbAmyA, left), and AmyS and TorA signal peptide (torA-BlAmyS, right) inserted into the pBbEB1c-rfp vector expressible Corynebacterium glutamicum according to an exemplary embodiment of the present disclosure.

In another aspect, the present disclosure provides a method for producing succinate from carbon dioxide, including fermenting starch by inoculating the strain producing succinate from carbon dioxide in a starch-containing medium. In an exemplary embodiment, the starch-containing medium may be a starch-containing minimal medium. Alternatively, it may be a medium containing a microalgal biomass lysate in which carbon dioxide is fixed. The strain producing succinate from carbon dioxide according to an exemplary embodiment of the present disclosure may be engineered from Corynebacterium glutamicum BL-1 as a mother strain for fermentation of microalgal biomass using carbon dioxide as a carbon source. After α-amylase is expressed in the cells of the mother strain, it may be secreted out of the cells by the TorA signal peptide, so that succinate can be produced using the starch-containing microalgal biomass as a carbon source.

Hereinafter, the present disclosure will be described in detail through examples. However, the following examples are for illustrative purposes only and it will be apparent to those of ordinary skill in the art that the scope of the present disclosure is not limited by the examples.

### Test Example 1

### Investigation of Growth of Wild-Type Corynebacterium glutamicum in Starch-Containing Medium

The following experiment was conducted to investigate growth of wild-type Corynebacterium glutamicum (Corynebacterium glutamicum ATCC13032) in a starch-containing medium.

0.5% starch (Sigma®) was added to CgXII minimal medium as a carbon source. The CgXII medium (pH 7) contained 20 g of (NH₄)₂SO₄, 5 g of urea, 1 g of KH₂PO₄, 1 g of K₂HPO₄, 0.25 g of MgSO₄.7H₂O, 10 mg of FeSO₄.7H₂O, 10 mg of MnSO₄.7H₂O, 1 mg of ZnSO₄.7H₂O, 0.2 mg of CuSO₄, 0.02 mg of NiCl₂.6H₂O, 0.2 mg of biotin, 0.42 mg of thiamine and 0.03 mg of protocatechuate (per liter).

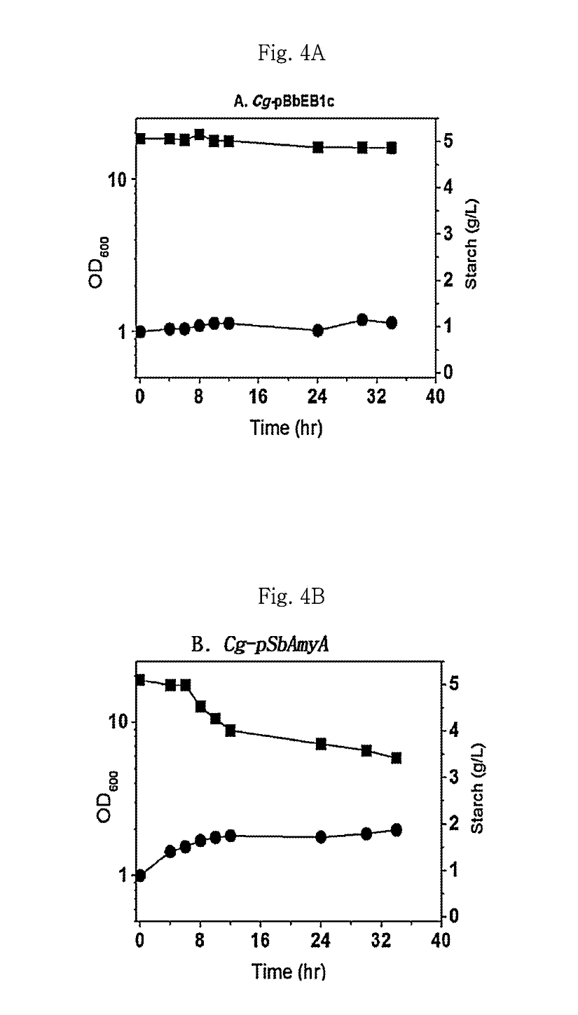

The strain was cultured in 50 mL of the medium in a 250-mL baffled Erlenmeyer flask at 30° C. for 56 hours after inoculation with an initial OD₆₀₀ of 1. As seen from FIG. 3, no growth of the wild-type strain was observed in the starch-containing medium.

Then, the change in starch concentration of the medium with time was quantitatively analyzed using Lugol's solution. Specifically, after preparing Lugol's solution (Sigma®), a sample was prepared by centrifuging at 14000 rpm for 10 minutes. Then, for quantitative analysis of starch concentration, a standard curve (0.05, 0.5, 1, 2 and 5 g/L) was constructed and a mixture of 1 mL of the supernatant of the sample and 0.1 mL of Lugol's solution was prepared in a 1.5-mL EP tube. The solution was diluted 10-fold because a lot of precipitate is formed as a result of binding between iodine in the Lugol's solution and starch if the starch concentration is 1 g/L of higher. The mixture containing the precipitate was transferred to a cuvette and absorbance was measured at 530 nm after vortexing. The measured OD value interpolated on the standard curve to determine the starch concentration. As seen from FIG. 3, no change in the starch concentration was observed in the medium.

### Test Example 2

### Preparation of Corynebacterium glutamicum Strain Growing Using Starch as Carbon Source

Screening of Target Genes for Allowing Strain to Use Starch as Carbon Source

For screening of genes allowing use of starch by encoding α-amylase, literature was searched. As a result of the literature search, the AmyA gene of Streptococcus bovis and the AmyS gene of Bacillus licheniformis, whose use in Corynebacterium glutamicum was not reported yet, were selected as target genes. The target genes AmyA and AmyS were codon-optimized for Corynebacterium glutamicum. The codon optimization was conducted using the DNA2.0's Gene Designer program. Data of the selected target genes are summarized in Table 1.

TABLE 1

| | Expressed | | |
|---|---|---|---|
| Gene name | protein | Strain name | Reference |
| AmyA | α-Amylase | Streptococcus bovis | Tateno et al., |
| | | | AMB 2007 |
| AmyS | α-Amylase | Bacillus licheniformis | Sibakov M., |
| | | | Eur. J. Biochem. |
| | | | 145: 567-572 |
| | | | (1984) |

Preparation of Succinate-Producing Strain

In order to secret α-amylase expressed by the target gene in cells out of the cells, the TorA signal peptide (SEQ ID NO 3) was attached to the base sequences of the codon-optimized AmyA gene and the codon-optimized AmyS gene. The resulting base sequences with the TorA signal peptide attached were named as torA-SbAmyA (SEQ ID NO 1) and torA-BlAmyS (SEQ ID NO 2).

Then, rfp (SEQ ID NO 7) was cleaved at the EcoRI/BamHI enzyme site of the pBbEB1c-rfp vector (SEQ ID NO 4) using the restriction enzymes EcoRI (SEQ ID NO 5, Fermentas®) and BamHI (SEQ ID NO 6, Fermentas®) and torA-SbAmyA and torA-BlAmyS were inserted respectively to the rfp site.

After transforming Corynebacterium glutamicum BL-1 as a mother strain according to an exemplary embodiment of the present disclosure and the Corynebacterium glutamicum ATCC13032 strain as a mother strain of the Corynebacterium glutamicum BL-1 respectively with torA-SbAmyA (SEQ ID NO 1) or torA-BlAmyS (SEQ ID NO 2), the genes were overexpressed through IPTG induction. Finally, each strain was cultured in the CgXII medium containing 0.5% starch (Sigma).

The Corynebacterium glutamicum BL-1 strains transformed with torA-SbAmyA (SEQ ID NO 1) or torA-BlAmyS (SEQ ID NO 2) were named as BL-1-pSbAmyA (Example 1) and BL-1-pBlAmyS (Example 2), respectively, and the Corynebacterium glutamicum ATCC13032 strains transformed with torA-SbAmyA and torA-BlAmyS were named as Cg-pSbAmyA and Cg-pBlAmyS, respectively. Separately from the above strains, the pBbEB1c vector with no torA-SbAmyA or torA-BlAmyS inserted was introduced to the Corynebacterium glutamicum BL-1 and Corynebacterium glutamicum ATCC13032 strains as controls.

### Test Example 3

### Investigation of Growth of Corynebacterium glutamicum Strain with Target Gene Introduced in Starch-Containing Medium

Before investigating the growth of the strain producing succinate from carbon dioxide according to an exemplary embodiment of the present disclosure in a starch-containing medium, the growth of Corynebacterium glutamicum ATCC13032 as a mother strain of Corynebacterium glutamicum BL-1 in which the torA-SbAmyA (SEQ ID NO 1) or torA-BlAmyS (SEQ ID NO 2) was inserted through genetic recombination was investigated as follows.

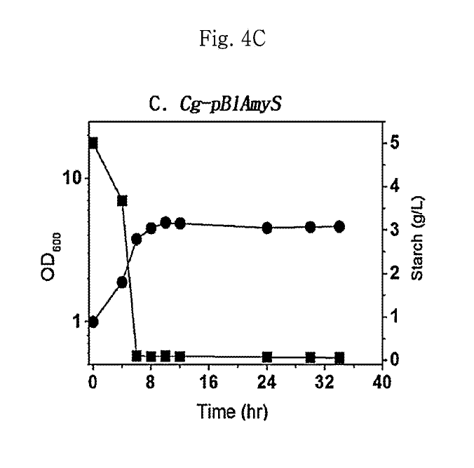

Specifically, Cg-pSbAmyA and Cg-pBlAmyS strains, obtained by transforming the Corynebacterium glutamicum ATCC13032 strain with torA-SbAmyA and torA-BlAmyS, respectively, and Cg-pBbEB1c strain in which only the pBbEB1c was inserted without the target gene were cultured in the CgXII medium containing 0.5% starch (Sigma®) and the OD₆₀₀ value was compared with time. As a result, Cg-pBlAmyS showed the highest OD value of 4.62 and Cg-pSbAmyA showed an OD value of 1.98. In contrast, cg-pBbEB1c showed an OD value of 1.14, indicating that it hardly grew. FIGS. 4a-4c show log plots of the OD₆₀₀ value with time. Based on the result, it is expected that the Corynebacterium glutamicum BL-1 strain and the strain producing succinate from carbon dioxide engineered according to the present disclosure will show similar growth behaviors.

The change in starch concentration of the medium with time was investigated in the same manner as described in Test Example 1 using Lugol's solution. The Cg-pBlAmyS which showed the fastest growth consumed most starch after 6 hours of culturing, whereas Cg-pSbAmyA showed no change in starch concentration after growth to some extent (see FIGS. 4a-4c).

### Test Example 4

### Investigation of Growth of Corynebacterium glutamicum BL-1 Strain with Target Gene Introduced and Production of Succinate in Starch- and Glucose-Containing Medium

The growth of the strain producing succinate from carbon dioxide according to an exemplary embodiment of the present disclosure in microalgal biomass was investigated as follows.

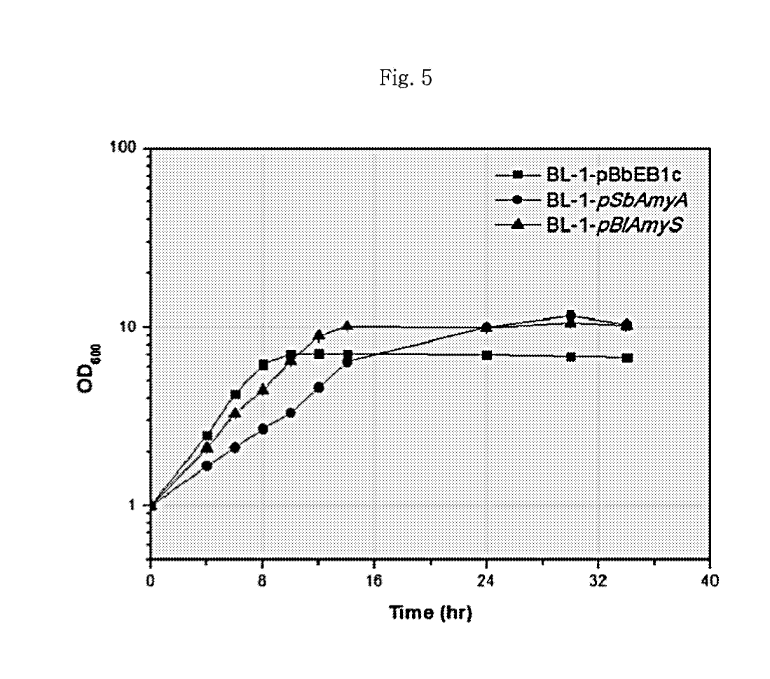

Specifically, BL-1-pSbAmyA (Example 1) and BL-1-pBlAmyS (Example 2) prepared by transforming the Corynebacterium glutamicum BL-1 strain with torA-SbAmyA and torA-BlAmyS, respectively, and BL-1-pBbEB1c (Comparative Example 1) prepared by inserting only pBbEB1c to Corynebacterium glutamicum BL-1 without the target gene were cultured in CgXII medium containing 0.5% starch (Sigma®) and 0.5% glucose. During the culturing, OD₆₀₀ value was compared with time. As a result, BL-1-pSbAmyA (Example 1) and BL-1-pBlAmyS (Example 2) showed very high final OD values of 10, whereas BL-1-pBbEB1c (Comparative Example 1) showed low growth rate of 30% with an OD value of 6.57 (see FIG. 5). It is because BL-1-pBbEB1c (Comparative Example 1) grew using only glucose, not starch, contained in the medium as a carbon source.

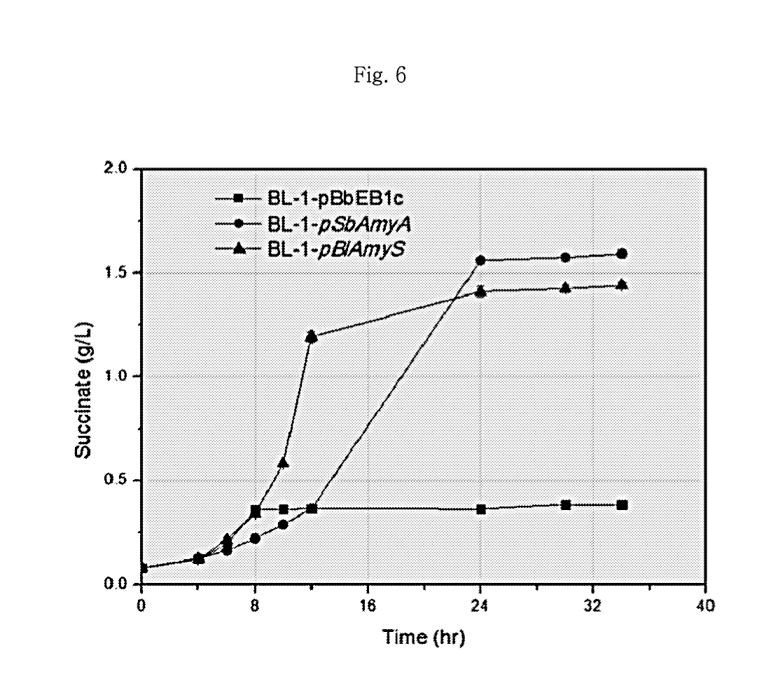

Also, the production of succinate in Examples 1-2 and Comparative Example 1 with time was investigated in the same manner as described in Test Example 1 using Lugol's solution. As a result, BL-1-pSbAmyA (Example 1) showed the highest succinate production of about 1.6 g/L and BL-1-pBlAmyS (Example 2) showed succinate production of about 1.44 g/L. In contrast, BL-1-pBbEB1c (Comparative Example 1) showed very low succinate production of about 0.47 g/L. From this result, it can be seen that the strains of Examples 1-2 according to the present disclosure used starch contained in the medium as a carbon source, unlike BL-1-pBbEB1c (Comparative Example 1) (see FIG. 6).

### Test Example 5

### Investigation of Growth of Corynebacterium glutamicum BL-1 Strain with Target Gene Introduced Target Gene and Production of Succinate in Medium Containing Microalgal Biomass

The growth behavior of the strain producing succinate from carbon dioxide according to an exemplary embodiment of the present disclosure in a medium containing microalgal biomass was investigated as follows.

Specifically, BL-1-pSbAmyA (Example 1) and BL-1-pBlAmyS (Example 2) prepared by transforming the Corynebacterium glutamicum BL-1 strain with torA-SbAmyA and torA-BlAmyS, respectively, and BL-1-pBbEB1c (Comparative Example 1) prepared by inserting only pBbEB1c to Corynebacterium glutamicum BL-1 without the target gene were cultured in CgXII medium containing 0.2% total sugar isolated from the green alga Chlamydomonas reinhardtii UTEX 90 sample as a microalgal biomass. The Chlamydomonas reinhardtii can be acquired from the Korean Collection for Type Cultures (KCTC) of the Korea Research Institute of Bioscience and Biotechnology (KRIBB). The microalgal biomass consisted of 60% carbohydrate and protein, lipid, etc. as the remainder. The carbohydrate consisted of 30% starch, the remaining 70% including D-glucose, L-fucose, L-rhamnose, D-arabinose, D-galactose, D-mannose, etc.

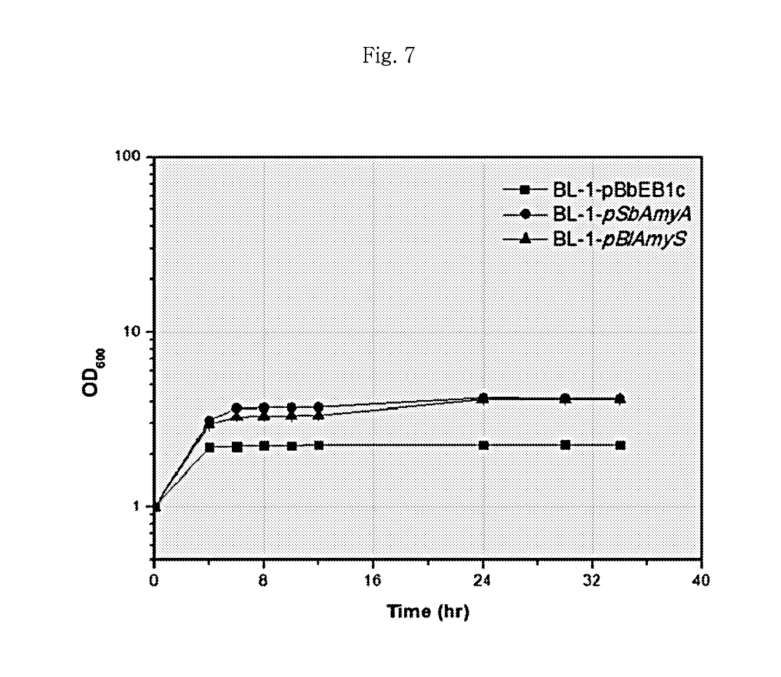

During the culturing, OD₆₀₀ value was compared with time. As a result, pBbEB1c-torA-SbAmyA (Example 1) and pBbEB1c-torA-BlAmyS (Example 2) showed high OD values of 4.1, whereas BL-1-pBbEB1c (Comparative Example 1) showed low growth rate of 50% with an OD value of 2.26 (see FIG. 7). It is because the strains according to the present disclosure grew using the starch contained in the microalgae as a direct carbon source, unlike Comparative Example 1.

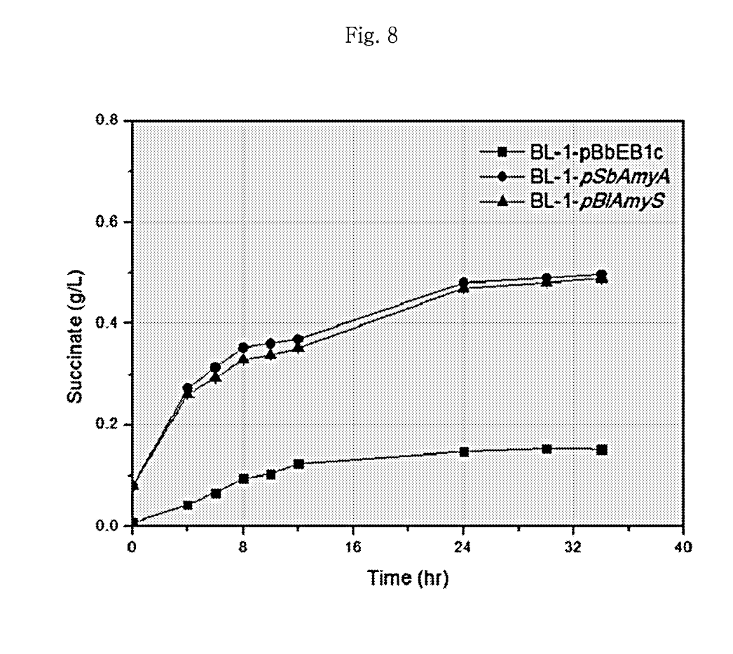

Also, the production of succinate in Examples 1-2 and Comparative Example 1 with time was investigated in the same manner as described in Test Example 1 using Lugol's solution. As a result, pBbEB1c-torA-SbAmyA (Example 1) showed the highest succinate production of about 0.5 g/L and pBbEB1c-torA-BlAmyS (Example 2) showed succinate production of about 0.49 g/L. In contrast, BL-1-pBbEB1c (Comparative Example 1) showed very low succinate production of about 0.15 g/L. It is because the strains of Examples 1-2 according to the present disclosure effectively used the starch contained in the medium as a carbon source, unlike BL-1-pBbEB1c (Comparative Example 1) (see FIG. 8).

Depository authority: Korean Collection for Type Cultures (KCTC)

Accession Nos.: KCTC 12585BP, KCTC 12586BP, KCTC 12587BP, KCTC 12588BP

Accession date: 2014, Apr. 29.

What is claimed is:

1. A strain producing succinate from carbon dioxide, selected from a group consisting of Corynebacterium glutamicum BL-1-pBlAmyS (KCTC 12585BP) and Corynebacterium glutamicum BL-1-pSbAmyA (KCTC 12587BP).

2. The strain producing succinate from carbon dioxide according to claim 1, wherein the strain producing succinate from carbon dioxide grows using starch accumulated in microalgal biomass that grows using carbon dioxide as a direct carbon source

3. The strain producing succinate from carbon dioxide according to claim 1, wherein the growth rate of strain producing succinate from carbon dioxide is 3 times or more higher in a starch-containing medium as compared to the mother strain Corynebacterium glutamicum BL-1 not including the gene AmyA or AmyS.

4. A method for preparing the strain producing succinate from carbon dioxide according to claim 1, comprising:
preparing torA-SbAmyA (SEQ ID NO 1) by attaching TorA signal peptide to the coding sequence of AmyA or preparing torA-BlAmyS (SEQ ID NO 2) by attaching TorA signal peptide to the coding sequence of AmyS;
inserting the torA-SbAmyA (SEQ ID NO 1) or the torA-BlAmyS (SEQ ID NO 2) to a vector expressible in Corynebacterium glutamicum BL-1 as a mother strain and genetically recombining the same with the mother strain Corynebacterium glutamicum BL-1; and
overexpressing the inserted torA-SbAmyA (SEQ ID NO 1) or torA-BlAmyS (SEQ ID NO 2) in the mother strain.

5. A method for producing succinate from carbon dioxide, comprising fermenting starch by inoculating the strain producing succinate from carbon dioxide according to claim 1 in a starch-containing medium.

6. The method for producing succinate from carbon dioxide according to claim 5, wherein the starch-containing medium is a medium containing a microalgal biomass lysate in which carbon dioxide is fixed.
